(19) United States
(12) Patent Application Publication
(10) Pub. No.: US 2024/0252215 A1
(43) Pub. Date: Aug. 1, 2024

(54) TARGETING AND INSERTION INSTRUMENT FOR ORTHOPEDIC IMPLANTS

(71) Applicant: Orthofix US LLC, Lewisville, TX (US)

(72) Inventors: Frank Larosa, Lewisville, TX (US); John Lovell, Lewisville, TX (US); Ethan Dias, Lewisville, TX (US)

(21) Appl. No.: 18/424,116

(22) Filed: Jan. 26, 2024

Related U.S. Application Data
(60) Provisional application No. 63/482,335, filed on Jan. 31, 2023.

(51) Int. Cl. A61B 17/70

(52) U.S. Cl. CPC A61B 17/7082

(57) ABSTRACT

Systems, methods, and devices for implanting a pedicle screw assembly using an inserter device. In some aspects, a device for inserting and implanting a screw into a bone of a patient may include a carrier, handle, and screwdriver. The carrier may include a first portion, a threaded second portion opposite the first portion, an opening extending from the first portion to the second portion and configured to receive a stylet, and a lock. The lock may be in communication with the opening and may be moved between a locked position and an unlocked position, where the lock engages the stylet to lock a position of the stylet relative to the carrier. The handle may have a threaded portion that engages the threaded second portion of the carrier. The screwdriver may couple to the handle, be configured to engage a threaded screw, and comprise a lumen configured to receive the stylet.

### CROSS-REFERENCE TO RELATED APPLICATIONS

The present applications claims the benefit of and priority to, U.S. Provisional Patent Application No. 63/482,335, filed Jan. 31, 2023, the entirety of which is incorporated by reference herein.

### TECHNICAL FIELD

The present disclosure relates generally to inserters and assemblies for implanting a pedicle screw assembly into a bone of a patient and methods of using the same.

### BACKGROUND

Many patients suffering from spinal deformities undergo minimally invasive spinal (MIS) surgery to stabilize and/or correct the deformity. MIS surgeries reduce trauma to the soft tissue and muscles surrounding the spine by accessing and placing devices in the spine via small incisions. For example, surgeons may implant pedicle screw assemblies into two or more vertebrae and connect each pedicle screw with a rod to stabilize the spinal levels. In order to minimize the footprint and ensure that the screw is delivered to the desired location, surgeons may first place guidewires or needles into the desired location before implanting the screw. The screw may then be implanted over the guidewire or needle. In this way, surgeons can avoid implanting the screw into the incorrect location where it may damage bone or nerves. Given the complexity of MIS surgeries, pedicle screw inserters have been developed that streamline and improve the process of implanting pedicle screw assemblies. Some of these inserters allow a surgeon to drive a needle or guidewire into the desired location, then implant a screw around the needle or guidewire. These inserters may accomplish this in a single step or in multiple steps. Moreover, these inserters may be a stand-alone instrument or may be used with another instrument.

However, in some cases, the needle or guidewire may be driven further into bone as the screw is screwed in around the needle or guidewire. Thus, improved inserters are needed to prevent the needle and/or screw from being unintentionally driven into an undesirable location while being easy for the surgeon to use.

### SUMMARY

The present disclosure describes systems, devices, and methods for targeting and inserting bone fixation implants and device, such as bone screws. Some embodiments according to the present disclosure include a device for implanting a screw into a bone of a patient that includes a carrier, a handle, and screwdriver. The carrier may comprise a first portion, a threaded second portion opposite the first portion, an opening extending from the first portion to the second portion, wherein the opening is configured to receive a stylet, and a lock. The lock may be in communication with the opening and may be moved between a locked position and an unlocked position, where the lock engages the stylet disposed within the opening to lock a position of the stylet relative to the carrier. The handle may have a threaded portion, where the second portion of the carrier can threadably engage with the threaded portion of the handle. The screwdriver may be coupled to the handle and may be configured to engage a threaded screw, wherein the screw driver comprises a lumen configured to receive the stylet.

In some embodiments, the first thread pitch of the threaded second portion of the carrier may be greater than a second threaded pitch of the threaded screw. In other embodiments, a first thread pitch of the threaded second portion of the carrier may be equal to a second threaded pitch of the threaded screw. In some embodiments, the stylet may comprise an elongate needle and a needle head, where the needle head may comprise a plurality of grooves. In some embodiments, the lock may engage at least one of the plurality of grooves when in the locked position. In some embodiments, the plurality of grooves may be arranged vertically along a length of the needle head. In some embodiments, each groove of the plurality of grooves may correspond to a length a tip of the needle extends past a bottom of the screw.

Some embodiments of the present disclosure include a device for implanting a screw into bone that includes a carrier, a stylet, a handle, a screwdriver, and a screw. The carrier may comprise a first portion, a threaded second portion, an opening extending from the first portion to the second portion, and a lock moveable between a locked position and an unlocked position. The stylet may have an elongate needle and a needle head, where at least a portion of the needle head is disposed within the carrier. The lock may engage the needle head in the locked position. The handle may have a third portion and a fourth portion, where the third portion of the handle may be threadably engaged with the threaded second portion of the carrier. The screwdriver may be coupled to the fourth portion of the handle and the screw may be coupled to the screwdriver.

In some embodiments, a first thread pitch of the threaded second portion of the carrier may be greater than a second threaded pitch of the threaded screw. In other embodiments, a first thread pitch of the threaded second portion of the carrier may be equal to a second threaded pitch of the threaded screw. In some embodiments, the stylet may be moveable within the carrier when the lock is in the unlocked position. In some embodiments, the needle head may comprise a plurality of grooves. In some embodiments, the lock may engage at least one of the plurality of grooves when in the locked position. In some embodiments, the plurality of grooves may be arranged vertically along a length of the needle head. In some embodiments, each groove of the plurality of grooves may correspond to a length a tip of the needle extends past a bottom of the screw.

Some embodiments of the present disclosure include a method of implanting a screw into a bone of a patient. The method may include the step of providing an instrument including a carrier, a handle, and a screwdriver. The carrier may include a first portion, a second portion, an opening extending from the first portion to the second portion, and a lock moveable between a locked position and an unlocked position. The handle may have a third portion and a fourth portion, where the third portion of the handle may be threadably engaged with the second portion of the carrier. The screwdriver may be coupled to the fourth portion of the handle and a screw may be coupled to the screwdriver. The method may also include the steps of providing a stylet having an elongate needle and a needle head and inserting the stylet into the instrument. The elongate needle may extend through the instrument and the screw such that a first length of a tip of the needle extends past a bottom of the screw and where at least a portion of the needle head may be disposed within the carrier and the lock is in a locked position such that the lock engages the needle head. The method may also include the steps of positioning the tip of the needle such that the tip contacts the bone, implanting the first length of the tip of the needle into the bone, implanting the screw into bone where the needle does not advance further into the bone, and removing the device from the implanted screw assembly.

In some embodiments, the method may also include the steps of adjusting, after implanting the tip of the needle into the bone, the device so that the needle extends a second length past the bottom of the screw and implanting a second length of the tip of the needle into the bone. In some embodiments, adjusting the device may include moving the lock of the carrier into an unlocked position. In some embodiments, the needle head of the stylet may comprise a plurality of grooves. In some embodiments, adjusting the device includes moving the lock into an unlocked position, moving the carrier relative to the stylet such that the lock aligns with a groove of the plurality of grooves, and moving the lock into a locked position, where the lock engages the groove of the plurality of grooves.

Additional aspects, features, and advantages of the present disclosure will become apparent from the following detailed description.

### BRIEF DESCRIPTION OF THE DRAWINGS

For a more complete understanding of the present disclosure and its advantages, reference is now made to the following description taken in conjunction with the accompanying drawings, in which like reference numbers indicate like features, and:

FIG. 1A is a perspective view of an assembled single-step screw inserter coupled to a pedicle screw assembly, according to aspects of the present disclosure.

FIG. 1B is an exploded view of the single-step screw inserter and pedicle screw assembly shown in FIG. 1A, according to aspects of the present disclosure.

FIG. 2A is a perspective view of a carrier, according to aspects of the present disclosure.

FIG. 2B is a vertical cross-section of the carrier shown in FIG. 3A, according to aspects of the present disclosure.

FIG. 2C is a horizontal cross-section of the carrier shown in FIG. 3A, according to aspects of the present disclosure.

FIG. 3 is a front view of a stylet, according to aspects of the present disclosure.

FIG. 4A is a perspective view of the handle shown in FIG. 4A, according to aspects of the present disclosure.

FIG. 4B is a vertical cross-section of a handle coupled to the carrier shown in FIGS. 3A-3C, according to aspects of the present disclosure.

FIG. 5A is a screwdriver coupled to a pedicle screw assembly, according to aspects of the present disclosure.

FIG. 5B is an exploded view of the screwdriver and pedicle screw assembly shown in FIG. 5A

FIG. 5C is a vertical cross-section of the screwdriver and pedicle screw assembly shown in FIG. 5A, according to aspects of the present disclosure.

FIG. 6 is a flow diagram of a method for implanting a screw using a single-step screw inserter, according to aspects of the present disclosure.

FIG. 7A is a front view of a screwdriver, according to aspects of the present disclosure.

FIG. 7B is a top view of a screwdriver with the lock housing removed, according to aspects of the present disclosure.

Although similar reference numbers may be used to refer to similar elements for convenience, it can be appreciated that each of the various example embodiments may be considered to be distinct variations.

### DETAILED DESCRIPTION

Exemplary embodiments will now be described hereinafter with reference to the accompanying figures, which form a part hereof, and which illustrate examples by which the exemplary embodiments, and equivalents thereof, may be practiced. As used in the disclosures and the appended claims, the terms “embodiment,” “example embodiment” and “exemplary embodiment” do not necessarily refer to a single embodiment, although it may and various example embodiments, and equivalents thereof, may be readily combined and interchanged, without departing from the scope or spirit of present embodiments. Furthermore, the terminology as used herein is for the purpose of describing example embodiments only and is not intended to be limitations of the embodiments. In this respect, as used herein, the term “plate” may refer to any substantially flat structure or any other three-dimensional structure, and equivalents thereof, including those structures having one or more portions that are not substantially flat along one or more axis. Furthermore, as used herein, the terms “opening,” “recess,” “aperture,” and equivalents thereof, may include any hole, space, area, indentation, channel, slot, bore, and equivalents thereof, that is substantially round, oval, square, rectangular, hexagonal, and/or of any other shape, and/or combinations thereof, and may be defined by a partial, substantial or complete surrounding of a material surface. Furthermore, as used herein, the term “in” may include “in” and “on,” and the terms “a,” “an” and “the” may include singular and plural references. Furthermore, as used herein, the term “by” may also mean “from,” depending on the context. Furthermore, as used herein, the term “if” may also mean “when” or “upon,” depending on the context. Furthermore, as used herein, the words “and/or” may refer to and encompass any and all possible combinations of one or more of the associated listed items.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, a description of a technology in the “Background” is not to be construed as an admission that technology is prior art to any invention(s) in this disclosure. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings herein.

The present disclosure is, in some aspects, directed to an inserter device for the use in orthopedic surgery. The inserter device 100 may be referred to as a single step inserter. The single step inserter may perform a single step or action or may perform multiple steps or actions. In some embodiments, a single step inserter may be used with or combined with another instrument. The inserter device 100 may include a carrier, a stylet, a handle, and a screwdriver. The carrier may be coupled to the top of the handle and the screwdriver may be attached to the bottom of the handle, such that openings in each part form a central lumen along the length of the assembly. The stylet may be inserted through the lumen of the carrier, handle, and screwdriver assembly such that the elongate needle of the stylet passes through the assembly and extends from the bottom of the screwdriver. A pedicle screw assembly may be coupled to the end of the screwdriver before the screwdriver is coupled to the handle, after the screwdriver is coupled to the handle, or after the stylet is inserted into the assembly. The screw of the pedicle screw assembly may have a lumen that aligns with the lumen of the carrier, handle, and screwdriver assembly. When the pedicle screw assembly is coupled to the inserter device, the elongate needle of the stylet may extend past the end of the screw. The distance the needle extends from the end of the screw may be set and manipulated using the carrier. The carrier may include a selective retention mechanism configured to engage the stylet and retain the stylet in one of a plurality of possible depths such that the amount of the needle protruding from the end of the screw can be controlled and modified.

The portion of the needle extending past the screw (i.e. the exposed length) may be driven into bone (e.g., by tapping with a hammer) until the distal tip of the screw contacts or engages the bone. The inserted portion of the needle may thus guide the insertion path of the screw into the bone. The inserter device may then be used to screw the shaft of the screw into bone around the needle. As the screw is being implanted into bone, the inserter device may either maintain the needle stationary in the bone or allow the needle to retract slightly. Thus, the inserter device prevents the needle from being driven further into bone when the screw is being screwed into the bone. Therefore, the inserter device may prevent the needle from advanced to an undesirable location, such as soft tissue, vertebral disc tissue, nerves, etc., thereby preventing damage to bone, tissue, and/or nerves in and around the spine.

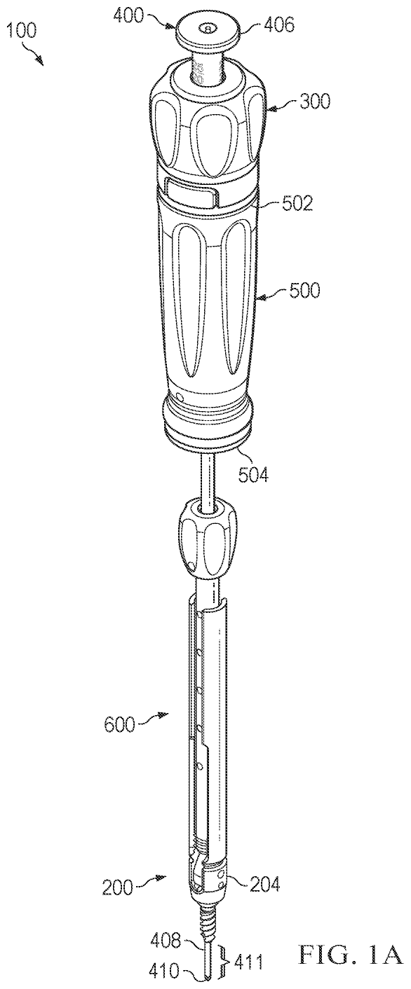

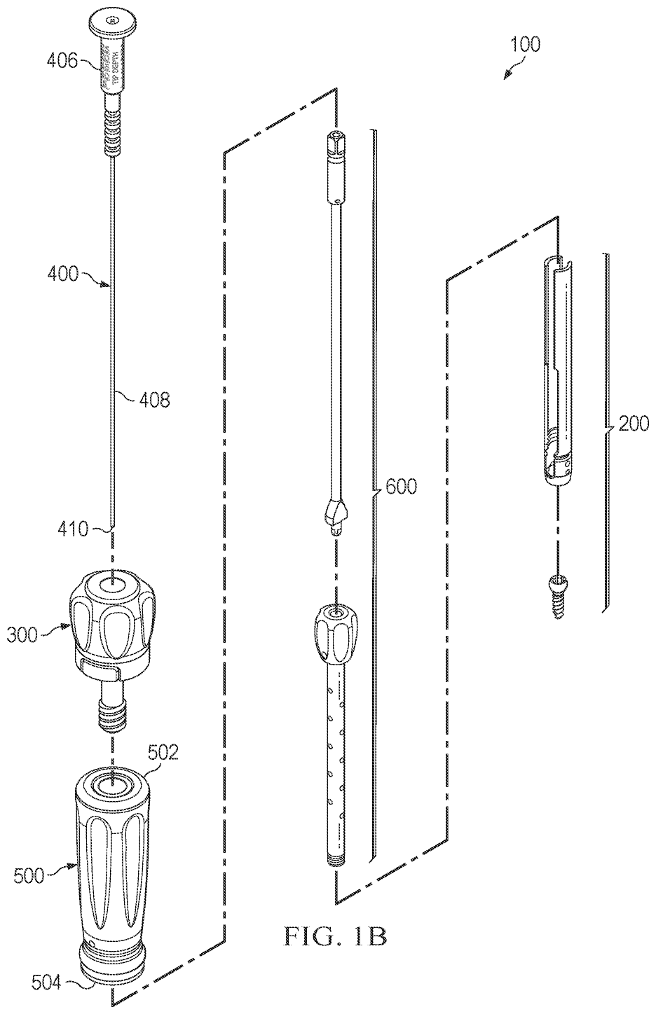

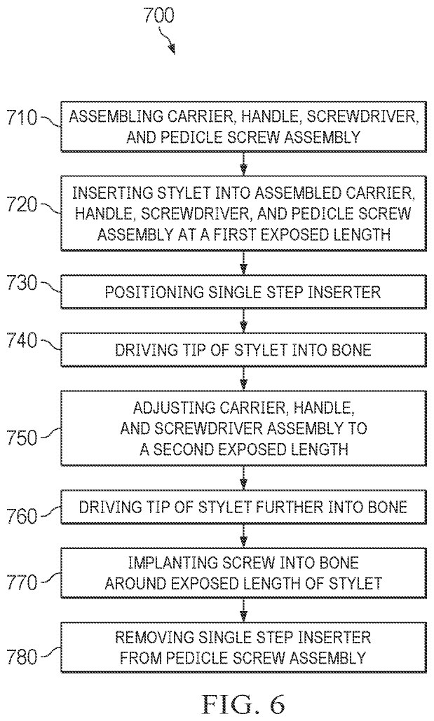

FIG. 1A is a perspective view of the assembled single step screw inserter coupled to a pedicle screw assembly according to one embodiment of the present disclosure. FIG. 1B is an exploded view of the single step screw inserter. The single step screw inserter comprises several parts, including a carrier 300, a stylet 400, a handle 500, and a screwdriver 600. The carrier 300 connects to the top 502 of the handle 500. The screwdriver 600 connects to the bottom 504 of the handle 500 opposite the carrier 300. The pedicle screw assembly 200 can be coupled to the bottom 604 of the screwdriver 600. As described in more detail below in reference to FIG. 6, the pedicle screw assembly 200 may be coupled to the screwdriver 600 either before or after the screwdriver 600 is coupled to the handle 500. The stylet 400 comprises a needle head 406 with an elongate needle 408 extending from the needle head 406. The stylet 400 may be inserted through the needle carrier 300 such that the elongate needle 408 extends through the carrier 300, the handle 500, and the screwdriver 600. When the pedicle screw assembly 200 is coupled to the screwdriver 600, the elongate needle 408 may also extend through the pedicle screw assembly 200 such that the tip 410 of the elongate needle 408 extends past the bottom of the pedicle screw assembly 200 by an exposed length 411.

The components of the inserter device 100 may be coupled in any appropriate way. For example, the carrier 300 may be threadably engaged with the handle 500. Similarly, the screwdriver 600 may be coupled to the handle 500 in any appropriate way. The screwdriver 600 may threadably engage the handle 500, may interact with a quick-release chuck in the handle 500, may have a friction-fit with the handle 500, may be secured into the handle 500 via a lock, may be adhered to the handle 500, or may use any other appropriate connection mechanism. Moreover, the stylet 400 may be coupled to the carrier 300 via, for example, a lock or push button on the carrier 300 that engages one or more grooves in the stylet 400. The stylet 400 may also threadably engage the carrier 300, may have a friction fit with the carrier 300, or may use any other appropriate connection mechanism. Additionally, the screwdriver 600 may threadably engage the pedicle screw assembly 200, may have a friction-fit with the pedicle screw assembly 200, may be secured into the pedicle screw assembly 200 via a lock, may be adhered to the pedicle screw assembly 200, or may use any other appropriate connection mechanism.

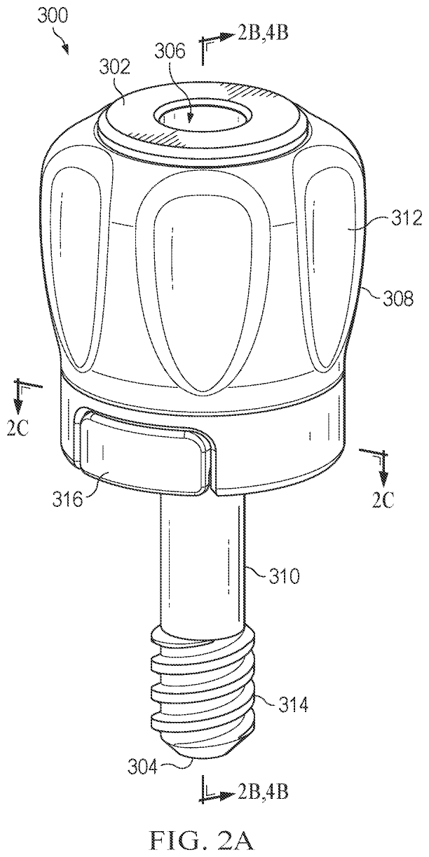

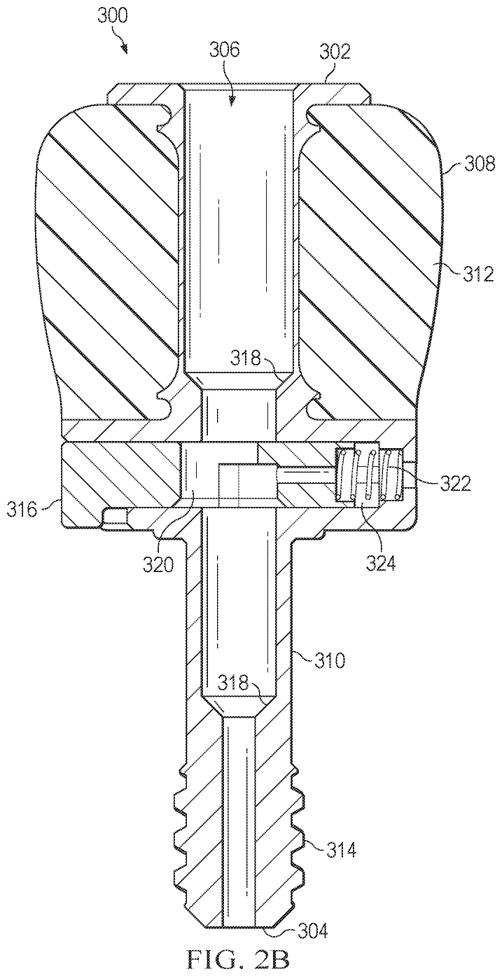

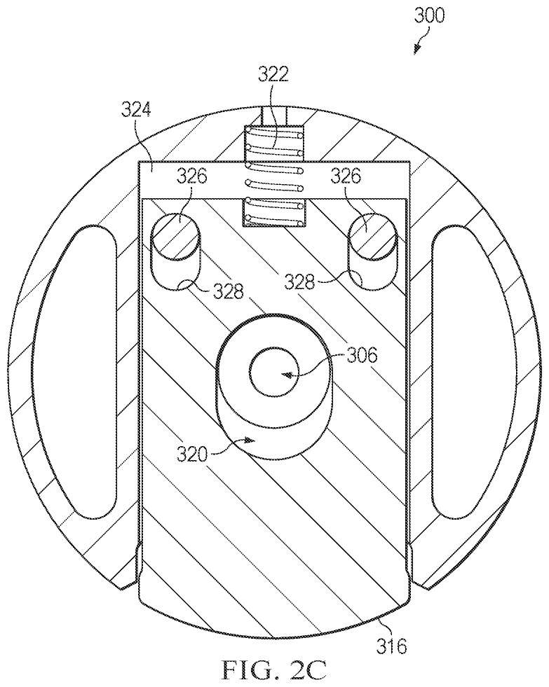

FIGS. 2A-5C illustrate various views of the components, assemblies, and sub-assemblies of the inserter. FIG. 2A shows a perspective view of the carrier 300 according to one embodiment. The carrier 300 has a top 302 and a bottom 304. There is an opening 306 that extends from the top 302 of the carrier 300 to the bottom 304. The carrier has an upper part 308 and a lower part 310. The upper part 308 may have a grip 312 on the exterior that may allow a surgeon to hold and maneuver the carrier 300. The grip 312 may be ergonomically designed to allow the surgeon to hold and maneuver the carrier 300 easily and comfortably. The grip may include features to improve grip and/or friction to improve maneuverability. For example, the grip 312 may comprise an elastomeric material such as silicone facilitating grip and comfort of the grip 312. The grip 312 may include notches, knurling, or other features in a metallic and/or plastic surface to increase friction. In some embodiments, the outer surface of the grip 312 may include vertical grooves to provide more catching surfaces for the user's hand.

The lower part 310 of the carrier 300 may be narrower than the upper part 308. The lower part 310 may have threading 314 that may extend partially (as shown in the illustrated embodiment) or completely along its length. As explained further below, the threading 314 may be configured to engage corresponding internal threading in the handle 500.

The carrier 300 also comprises a push button 316, which may be referred more generally as a lock or locking feature. In the illustrated embodiment, the push button 316 is located on the bottom of the upper part 308 of the carrier 300. However, the push button 316 may be located in any appropriate spot including the top or middle of the upper part 308 or the top, middle, or bottom of the lower part 310.

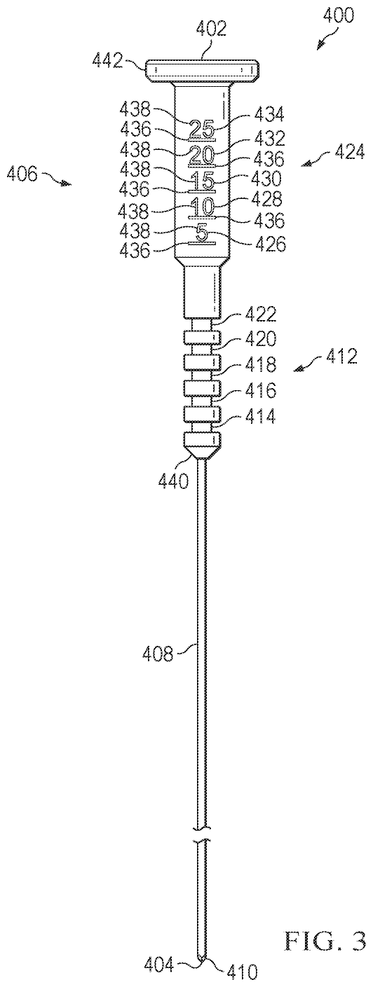

FIG. 2B shows a vertical cross-section of the carrier 300 along the 2B-2B line shown in FIG. 3A. FIG. 2C shows a horizontal cross-section of the carrier 300 along the 2C-2C line shown in FIG. 3A. The opening 306 of the carrier 300 narrows from the top 302 to the bottom 304. In the illustrated embodiment, the opening 306 has discrete tapering surfaces 318 where it narrows. However, in other embodiments, the opening may taper or may not narrow at all.

The push button 316 extends through the carrier 300 from one side and passes through the opening 306. The push button 316 has an aperture 320. The push button 316 is able to slide within the carrier 300 such that the aperture 320 can align with the opening 306 in a first (i.e. unlocked) position and can be offset from the opening 306 in a second (i.e. locked) position.

There is a biasing mechanism 322 that biases the push button 316 towards the locked position. In this example, the biasing mechanism 322 is a spring located in a spring compartment 324 of the lower part 310 of the carrier 300; however, any appropriate biasing mechanism may be used. The spring 322 contacts the push button 316 and pushes it towards the opposite side of the carrier 300, and thus pushes the aperture 320 out of alignment with the opening 306. However, the surgeon can press the push button 316 to move the push button 316 towards the spring 322 so that the aperture 320 is aligned with the opening 306. In this way, the surgeon can move the push button 316 into the unlocked position by pressing on the push button 316. The push button 316 may comprise a locking surface configured to engage a corresponding groove or slot on the stylet 400, as further explained below. In the default, or resting position, the locking surface, which may be referred to as a detent, may be biased to engage the groove or slot on the stylet 400 such that the stylet 400 is locked in at least the longitudinal direction with respect to the carrier 300. Depressing the push button 316 inward may cause the locking surface of the push button 316 to disengage the groove or slot so that the stylet 400 can be either advanced further into the opening 306 or retracted from the opening 306.

In some embodiments, like the illustrated embodiment, the carrier 300 may have one or more pins 326 that fit within one or more slots 328 in the push button 316. Each pin 326 may extend into the slot 328. In the illustrated embodiment, the pins 326 may be located in the spring compartment 324. However, in other embodiments the pins 326 may be located in any part of the carrier 300 that contacts the push button 316. Moreover, the pins 326 may be vertical as shown or may be horizontal. As the push button 316 moves between a locked and unlocked position, the slots 328 may slide along the pins 326 to prevent rotation of the push button 316 and to limit the amount that the push button 316 can move back and forth.

FIG. 3 shows a front view of the stylet 400 according to some embodiments of the present disclosure. The stylet 400 has a needle head 406 at the top 402 of the stylet 400 and an elongate needle 408 at the bottom 404 of the stylet 400. In some embodiments, the needle head 406 may comprise several grooves. In the illustrated embodiment, the lower part 412 of the needle head 406 has five grooves 414, 416, 418, 420, 422. However, in other embodiments, the needle head 406 may comprise any number of grooves, including, for example, 1, 2, 3, 4, 5, 10, 15, or 20 grooves. The grooves may be any distance or increment apart, including, for example, 1 mm, 2 mm, 3 mm, 4 mm, 5 mm, 8 mm, 10 mm, or 15 mm. In some embodiments, the grooves are not the same distance or increment apart. For example, the first and second groove may be 1 mm apart and the second and third groove may be 2 mm apart. There may be any combination of any distance between the grooves. The grooves 414, 416, 418, 420, 422 may correspond to the exposed length 411 that the tip 410 of the elongate needle 408 extends from the bottom of the pedicle screw assembly 200.

The needle head 406 may have an upper part 424. In some embodiments, the upper part 424 of the needle head 406 has a series of measurement indicators corresponding to the amount the elongate needle 408 extends from the bottom of the pedicle screw assembly 200. In the illustrated embodiment, the needle head 406 has five measurement indicators 426, 428, 430, 432, 434 that correspond to the exposed length 411 that the tip 410 of the elongate needle 408 extends from the bottom of the pedicle screw assembly 200. In the illustrated embodiment, measurement indicator 426 corresponds to an exposed length 411 of 5 millimeters (mm), measurement indicator 428 corresponds to an exposed length 411 of 10 mm, measurement indicator 430 corresponds to an exposed length 411 of 15 mm, measurement indicator 432 corresponds to an exposed length 411 of 20 mm, and measurement indicator 434 corresponds to an exposed length 411 of 25 mm. However, in other embodiments, the needle head 406 may have any number of measurement indicators and the measurement indicators may correspond to any exposed length of the elongate needle 408. For example, the needle head may have 1, 2, 3, 4, 5, 10, 15, or 20 measurement indicators and those measurement indicators may correspond to any one of 1 mm, 2 mm, 3 mm, 4 mm, 5 mm, 10 mm, 15 mm, 20 mm, 30 mm, 40 mm, 50 mm, 60 mm, 70 mm, 80 mm, 90 mm, or 100 mm. As shown in the illustrated embodiment, the measurement indicators 426, 428, 430, 432, 434 may comprise a line 436 and the value 438 of the corresponding exposed length 411.

The grooves 414, 416, 418, 420, 422 may correspond to the measurement indicators 426, 428, 430, 432, 434 and, thus, may allow a surgeon to control the exposed length 411 of the elongate needle 408.

The stylet needle 408 may be inserted through the top 302 of the opening 306 of the carrier 300 such that the elongate needle 408 extends through the bottom 304 of the carrier 300. The stylet 400 may have an inclined surface 440 on the bottom of the lower part 412 of the needle head 406. As the stylet 400 is inserted into the carrier 300, the surgeon may press down on the stylet 400, which causes the inclined surface 440 to press against the aperture 320 of the push button 316. This force may move the push button 316 such that the aperture 320 aligns with the opening 306 so that the stylet 400 will move down and the push button 316 will align with the first groove 414 of the lower part 412 of the needle head 406. In other words, the surgeon may be able to move the push button 316 into an unlocked position by pressing down on the stylet 400 instead of by pressing the push button 316. When the push button 316 aligns with the first groove 414, the push button 316 may move back into a locked position and contact the first groove 414. This locks the stylet 400 into the carrier 300 such that the stylet 400 and the carrier 300 cannot move relative to each other.

When the stylet 400 is locked into the carrier 300, the exposed length 411 of the elongate needle 408 will correspond to the exposed length 411 associated with the first groove 414 that contacts the push button 316. The measurement indicator 426 associated with that groove will be aligned with the top 302 of the carrier 300. Thus, in the illustrated embodiment, the first groove 414 corresponds to an exposed length 411 of 5 mm and the line 436 of the 5 mm measurement indicator 426 may align with the top 302 of the carrier 300.

In order to move the carrier 300 up or down the stylet 400, the surgeon can press on the push button 316 to move it into an unlocked position. When in the unlocked position, the stylet 400 can be moved up or down. Thus, the surgeon can adjust the exposed length 411 of the elongate needle 408 by aligning the measurement indicator corresponding to the desired exposed length 411 with the top 302 of the carrier, thereby aligning the groove corresponding to the desired exposed length 411 with the push button 316. The stylet 400 may have a wider upper end 442 on the top 402 to prevent the needle head 406 of the stylet from passing through the carrier 300, thus allowing the surgeon to remove the stylet 400 from the carrier 300. Additionally, the discrete narrowing or tapering surfaces 318 within the opening 306 of the carrier 300 may also prevent the stylet 400 from moving deeper within the carrier 300. However, in some embodiments, any appropriate means for preventing the stylet 400 from moving deeper within the carrier 300 may be used. In other embodiments, there may be no means of preventing the stylet 400 from moving deeper within the carrier 300.

In other embodiments, the stylet 400 may not have an inclined surface 440 on the bottom of the lower part 412 of the needle head 406. Instead, when the stylet is inserted into the carrier 300, the surgeon may press on the push button 316 to move it into an unlocked position and thereby allowing the surgeon to align the push button 316 with the desired groove. In some embodiments, the grooves may not extend completely around the needle head 406 and may instead extend only partially around the needle head 406. In other embodiments, the needle head 406 may not have an upper part 424 and a lower part 412 and may instead only have one part or may have more than two parts.

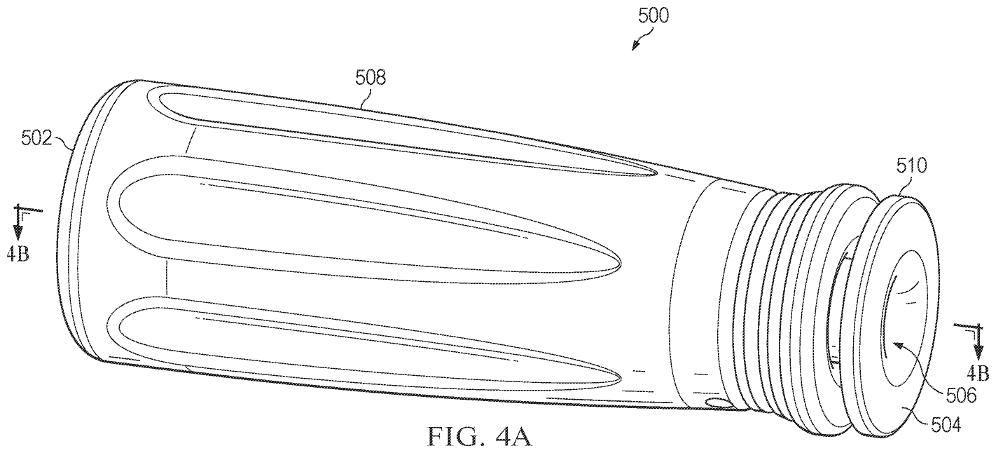

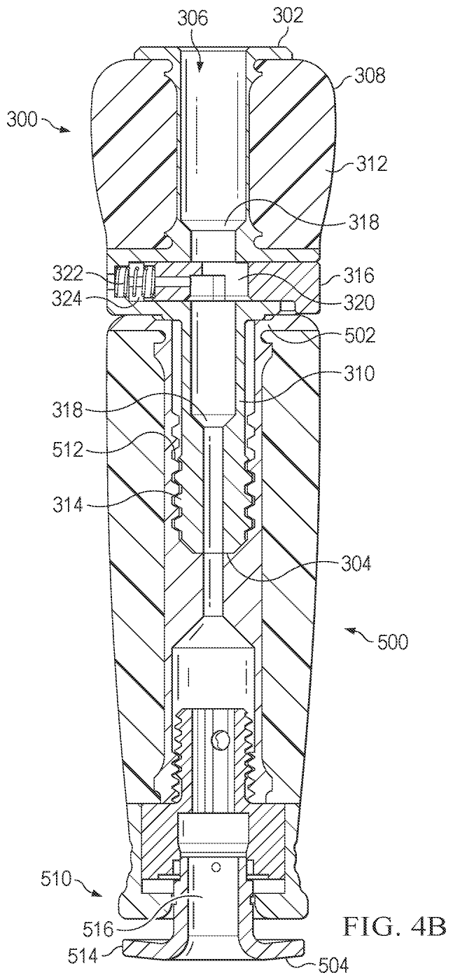

FIG. 4A illustrates the handle 500 according to one implementation. The handle 500 has a top 502 and a bottom 504. There is an opening 506 that extends from the top 502 of the handle 500 to the bottom 504. The handle 500 may have a grip 508 that may allow a surgeon to hold and maneuver the handle 500. The grip 508 may be ergonomically designed to allow the physician or other health care provider to hold and maneuver the handle 500 easily and comfortably.

The handle 500 includes a screwdriver connection interface 510. In the illustrated embodiment, the interface 510 comprises a quick release chuck at the bottom 504 of the handle 500 that couples to the screwdriver 600. In some embodiments, there may not be a quick release chuck and may instead use any appropriate connector for coupling the handle 500 and the screwdriver 600. For example, the handle 500 and screwdriver 600 may be connected using an internal or external threading, a magnetic connection, a snap-fit connection, a friction element, a lock, or any other appropriate connection. The interface 510 may be configured to lock the screwdriver 600 to the handle 500 rotationally, such that the physician may control the screwdriver 600 by rotating the handle 500.

FIG. 4B shows a cross section of the carrier 300 of FIGS. 2A-2C coupled to the handle 500 shown in FIG. 4A. The wall of the opening 506 of the handle 500 has a threaded portion 512. The threaded portion 512 may start at the top 502 of the opening 506 and extend partially or completely down the length of the opening 506. The threading 314 on the lower part 310 of the carrier 300 may threadably engage the threaded portion 512 of the handle 500, thus coupling the carrier 300 to the handle 500. In other embodiments, the carrier 300 may be coupled to the handle 500 by any appropriate means. For example, the carrier 300 may snap-fit into the handle 500, the handle 500 may have a lock that engages the carrier 300, the carrier 300 may have a friction element that interacts with the handle 500, or any other appropriate connection.

When the carrier 300 and the handle 500 are coupled, the opening 306 of the carrier 300 aligns with the opening 506 of the handle 500 such the elongate needle 408 of the stylet 400 can pass through both openings 306, 506 when it is inserted into the carrier 300.

The handle 500 has an interface 510 (e.g., quick release chuck) for connecting the screwdriver 600 to the handle 500. The interface 510 may have a connector 514 having an opening 516 that aligns with the opening 506 of the handle 500 and through which the top of the screwdriver 600 may be inserted. The connector 514 may move up as the screwdriver 600 is pushed up through the opening 516. There may be ball bearings (not shown) disposed in the opening 516 of the interface 510 that engage a groove of the screwdriver 600 as described in more detail below. The interface 510 may comprise a subassembly that can be removably connected or coupled to the remaining subassembly of the handle 500. For example, in the illustrated embodiment, the interface 510 comprises a subassembly that forms a threaded connection with the rest of the handle 500. In other embodiments, the interface 510 may be permanently or semi-permanently attached to the rest of the handle 500. For example, the interface 510 may be coupled to the rest of the handle 500 by one or more of an adhesive, welding, rivets, pins, set screws, and/or any other suitable means of connection.

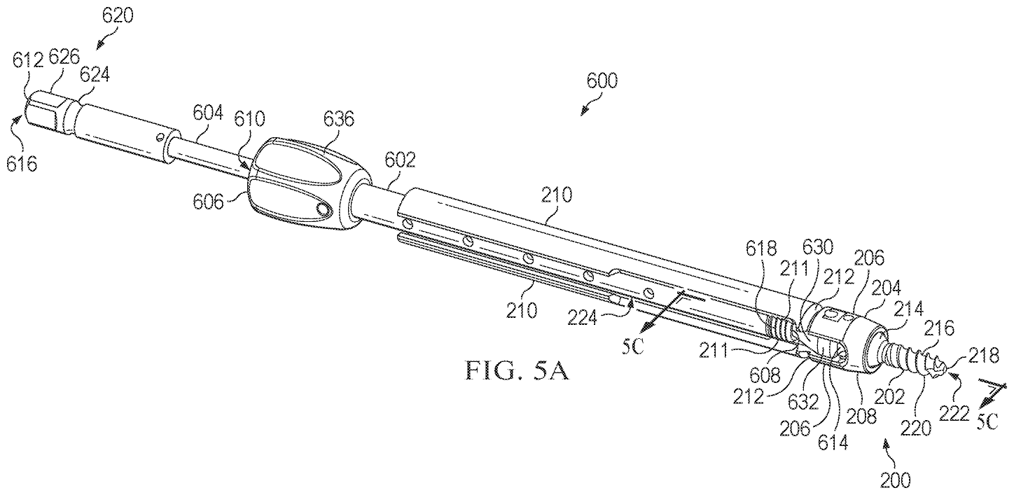

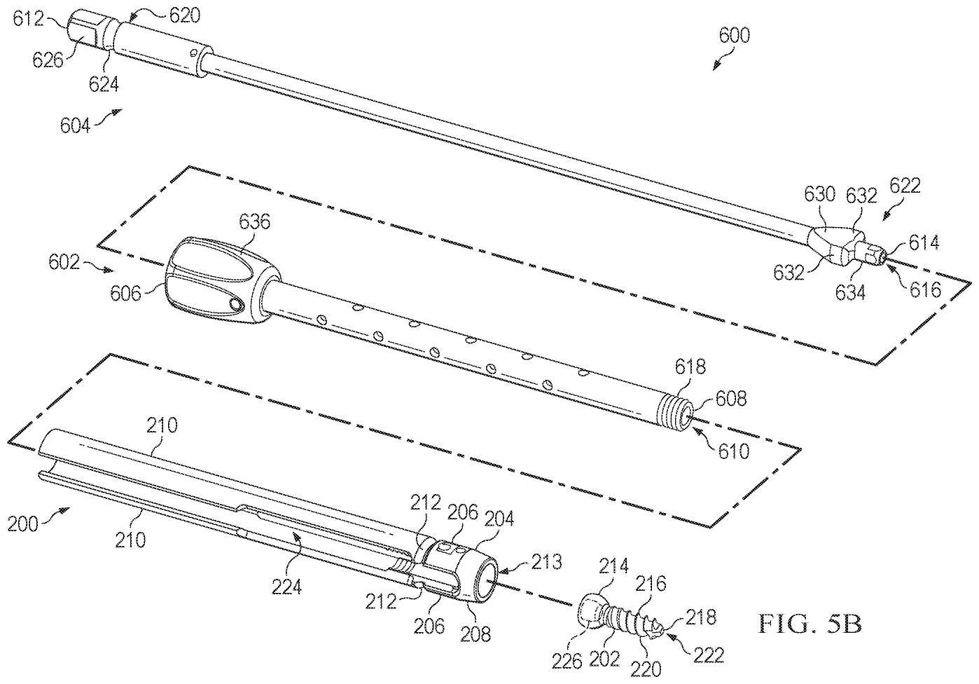

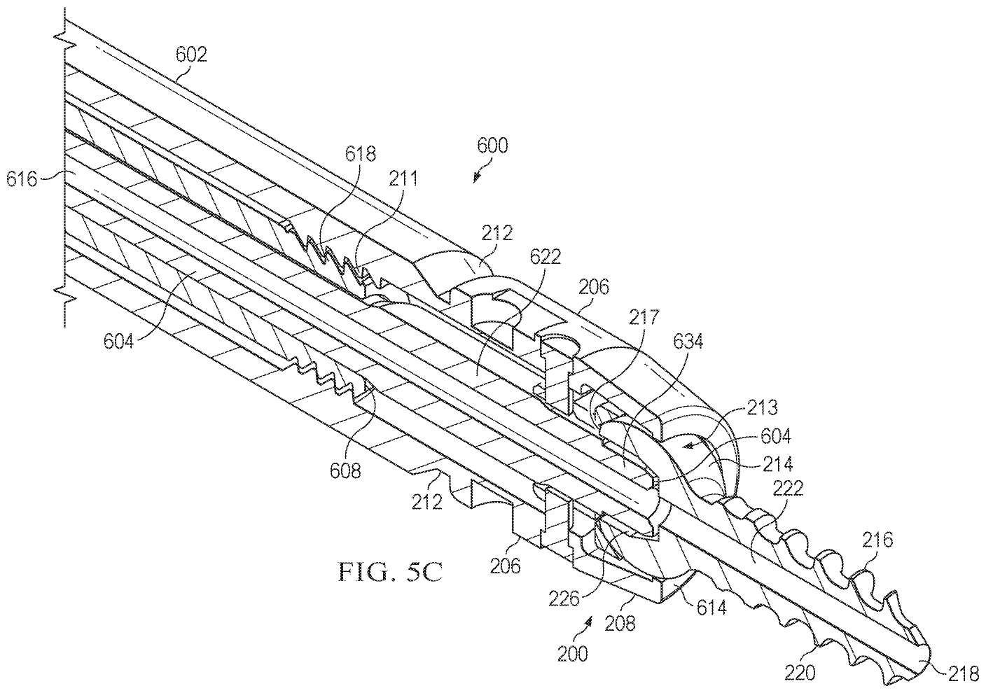

FIG. 5A illustrates one embodiment of an assembled screwdriver 600 coupled to a pedicle screw assembly 200 according to an embodiment of the present disclosure. FIG. 5B shows an exploded view of the screwdriver 600 and the pedicle screw assembly 200. FIG. 5C shows a cross section of the bottom of the screwdriver 600 coupled to the pedicle screw assembly 200 along the line shown in FIG. 5A. The pedicle screw assembly 200 can include a screw 202 and a tulip 204. The tulip 204 may have two arms 206 that extend upwards from a base 208 of the tulip 204. The arms 206 can have extensions, which may be referred to as towers 210. There may be a thinner amount of material forming the intersection 212 between each tower 210 and arm 206 so that the towers 210 can be removed from the arms 206. For example, the towers 210 can be broken from the arms 206 at the intersection 212, a tool can be used to cut or remove the intersection 212, or the towers 210 can be removed from the arms 206 in any other appropriate way. Moreover, there may be a threading 211 that extends along the towers 210 and the arms 206 facing the inner portion of the tulip 204. In some embodiments, the threading 211 is on either the towers 210 or the arms 206. In other embodiments, the tulip 204 may have another connection mechanism including, for example, a groove, a lock, a compressible ring, or any other appropriate mechanism.

Additionally, the tulip 204 can have an opening 213 from the top of the tulip 204 to the bottom so that the opening 213 extends between the towers 210 and arms 206 and through the base 208.

The screw 202 may comprise a head 214 and a shaft 216. The screw 202 may be top-loaded or bottom-loaded into the opening 213 of the tulip 204 until the head 214 is seated in the base 208. The bottom of the head 214 may be spherically shaped so that the screw 202 can pivot, rotate, or move when seated within the tulip 204. Additionally, there can be an engagement feature 226 on the top 217 of the head 214 of the screw 202. The engagement feature 226 may be any appropriate shape. For example, in the illustrated embodiment, the engagement feature 226 has four sides creating a square shape. In some embodiments, the engagement feature 226 may be triangular, hexagonal, round, linear, or any other appropriate shape. In the illustrated embodiment, the engagement feature 226 is a hole, but in other embodiments, the engagement feature may be a projection, tabs, threaded, or any other appropriate shape.

The shaft 216 of the screw 202 extends from the bottom of the head 214 and may come to a point at the bottom 218 of the screw 202. There may be threading 220 that extends completely or partially down the length of the shaft 220. In some embodiments, the thread pitch of the threading 220 of the screw 202 may be equal to the thread pitch of the threading 314 on the lower part 310 of the carrier 300. In other embodiments, the thread pitch of the threading 314 on the lower part 310 of the carrier 300 may be greater than the thread pitch of the threading 220 of the screw 202. Moreover, the screw 202 may include a lumen 222 (not shown in FIG. 5A) that passes from the top 217 of the screw 202 to the bottom 218 of the screw 202.

The screwdriver 600 comprises a tulip connector 602 and a screw connector 604. The tulip connector 602 has a top 606 and a bottom 608 with an opening 610 extending from the top 606 to the bottom 608. Similarly, the screw connector 604 also has a top 612 and a bottom 614 with a lumen 616 extending from the top 612 to the bottom 614. The screw connector 604 fits through the opening of the tulip connector 602. Thus, when the screwdriver 600 is coupled to the handle 500 and the stylet 400 is inserted through the carrier 300, the elongate needle 408 of the stylet 400 can pass through the opening 306 of the carrier 300, the opening 506 of the handle, and the lumen of the screw connector 604. When the pedicle screw assembly 200 is coupled to the screwdriver 600, the lumen 222 of the screw 202 aligns with the lumen 616 of the screw connector 604 so that the elongate needle 408 of the stylet 400 can pass from the lumen 616 of the screw connector 604 through the lumen 222 of the screw 202. In some embodiments, an exposed length 411 of the elongate needle 408 may extend past the bottom 218 of the screw 202.

The tulip connector 602 may also have a grip 636 at the top 606 that may allow a surgeon to hold and maneuver the screwdriver 600. The grip 636 may be ergonomically designed to allow the surgeon to hold and maneuver the screwdriver 600 easily and comfortably.

The tulip connector 602 may also have a threaded portion 618 at the bottom 608. The threaded portion 618 of the tulip connector 600 may engage with the threading 211 on the towers 210 of the tulip 204 of the pedicle screw assembly 200. In this way, the tulip connector 602 may couple to the tulip 204 and thus the pedicle screw assembly 200. In some embodiments, the threaded portion 618 of the tulip connector 602 may engage with the threading on the arms 206 of the tulip 204. However, the tulip connector 602 may couple to the tulip 204 by any appropriate means. For example, the tulip connector 602 and tulip 204 may have a friction-fit or may snap together, the tulip connector 602 may expand to connect with the tulip 204, there may be a lock to connect the tulip connector 602 to the tulip 204, or the tulip connector 602 and the tulip 204 may connect in any other appropriate way. The tulip connector 602 may be attached to tulip 204 by rotating the grip 636 such that the tulip connector 602 rotates independently of the pedicle screw assembly 200 and the screw connector 604. In this way, the threading 618 on the tulip connector 602 engages the threading 211 on the towers 210, securing the tulip connector 602 to the tulip 204. This process is described in more detail below.

The screw connector 604 has a handle attachment 620 at the top 612. The handle attachment 620 has a non-cylindrical shaft 626 or head at the top 612 of the screw connector 604. In the illustrated embodiment, the non-cylindrical shaft 626 is square-shaped having 4 sides. However, the non-cylindrical shaft 626 may be any appropriate shape including, for example, hexagonal, triangular, ovular, or any other appropriate shape. In some embodiments, the shaft may be cylindrical.

There may be a groove 624 disposed below the non-cylindrical shaft 626. The handle attachment 620 may be inserted into the bottom 504 of the handle 500 through the opening 516 of the connector 514 of the quick release chuck 510. As the screw connector 604 is pressed upwards, the surfaces of the non-cylindrical shaft 626 may move the bearing balls of the quick release chuck 510 outward. When the groove 624 aligns with the bearing balls, the bearing balls move inward and engage the groove 624 of the screw connector 604. This couples the screwdriver 600 to the handle 500. The non-cylindrical shaft 626 may prevent the screw connector 604 from rotating relative to the handle 500, thereby allowing the screwdriver 600 to rotate with the handle 500.

To remove the screw connector 604 from the quick release chuck 510, the surgeon can pull on the screw connector 604. The downward force can move the bearing balls outward, thus releasing the handle attachment 620 from the quick release chuck 510.

The screw connector 604 may also include a screw attachment 622 at the bottom 614 of the screw connector 604 opposite the handle attachment 620. The screw attachment 622 has a winged portion 630 with two extensions 632 that extend outwardly from the center of the screw connector 604. The winged portion 630 may also include a central projection 634 extending along the longitudinal axis of the screw connector 604. The central projection 634 may have an end shape that corresponds to the shape of the engagement feature 226 on the head 214 of the screw 202. In this embodiment, the central projection 634 is a four-sided (square-shaped). However, in other embodiments, the central projection 634 may be any other appropriate shape, including, for example, triangular, hexagonal, round, linear, or any other appropriate shape. Moreover, the in some embodiments, there is not a central projection 634, but a central hole which is shaped to engage a projection engagement feature 226. In some embodiments, the central projection 634 may not correspond to the shape of the engagement feature 226, but may still engage the engagement feature 226. The opening 616 of the screw connector 604 may pass through the central projection 634.

The winged portion 630 may be aligned with the towers 210 of the tulip 204 such that the extensions 632 slide between the open U-shaped channel 224 between the towers 210. The winged portion 630 may slide down the towers 210 until the central projection 634 engages the engagement feature 226 on the head 214 of the screw 202.

Thus, the extensions 632 and the central projection 634 may engage the pedicle screw assembly 200 such that as the screwdriver 600 rotates, the pedicle screw assembly 200 rotates with it. Thus, when the handle 500 is rotated, the screwdriver 600 and the pedicle screw system 200 attached to it are rotated along with the handle 500. Therefore, by rotating the handle 500, the screw 202 may be screwed into bone. This process is described in more detail below.

FIG. 6 illustrating a method 700 of using the inserter device 100 to implant a pedicle screw assembly 200 into bone. At step 710 of the method, the carrier 300, handle 500, screwdriver 600, and pedicle screw assembly 200 are assembled. The carrier 300 may be attached to the handle 500 by inserting the lower part 310 of the carrier 300 through the top 502 of the opening 506 of the handle 500 and screwing the threading 314 on the lower part 310 into the threading portion 512 of the opening 506.

The pedicle screw assembly 200 may be attached to the screwdriver 600 by aligning the extensions 632 on the winged portion 630 of the screw connector 604 with the channel 224 between the towers 210 of the tulip 204, then sliding the extensions 632 down the channel 224 until the central projection 634 of the screw connector 604 contacts and engages with the engagement feature 226 of the head 214 of the screw 202. The surgeon can then tighten the attachment between the screwdriver 600 and the pedicle screw assembly 200 by rotating the grip 636 of the tulip connector 602, thereby engaging the threaded portion 618 of the tulip connector 602 with the threading 211 on the towers 210 of the tulip 204.

The screwdriver 600 may be attached to the handle 500 by pressing the handle attachment 620 of the screwdriver 600 up into the quick release chuck 510 of the handle 500 until the ball bearings engage the groove 624 of the handle attachment 620.

In some embodiments, the pedicle screw assembly 200 may be attached to the screwdriver 600 before the screwdriver 600 is attached to the handle 500. However, in some embodiments, the screwdriver 600 may be attached to the handle 500 before the pedicle screw assembly 200 is attached to the screwdriver 600. Similarly, the carrier 300 may be attached to the handle 500 before or after the screwdriver 600 is attached to the handle 500.

Once the carrier 300, handle 500, screwdriver 600, and pedicle screw assembly 200 are assembled, the assembly forms a central lumen from the top 302 of the carrier 300 to the bottom 218 of the screw 216.

Referring back to FIG. 6, at step 720, the stylet 400 is inserted into the assembly of the carrier 300, handle 500, screwdriver 600, and pedicle screw assembly 200. The needle 408 of the stylet 400 is inserted through the central lumen from top 302 of the carrier 300 to the bottom 218 of the screw 216. Thus, the needle 408 extends through the carrier 300, handle 500, screwdriver 600, and pedicle screw assembly 200. The tip 410 of the needle 408 may extend past the bottom 218 of the screw 216 such that there is an exposed length 411 of needle 408 extending from the bottom 218 of the pedicle screw assembly 200.

The biasing mechanism 322 of the push button 316 of the carrier 300 biases the push button 316 into the locked position such that it narrows the opening 306 of the carrier 300. In the locked position, the needle 408 can pass through the opening 306 in the carrier 300, but not the needle head 406. Thus, when the needle 408 is inserted through the central lumen, the inclined surface 440 on the bottom of the lower part 412 of the needle head 406 contacts the push button 316 in the locked position. In some embodiments, the surgeon may apply additional pressure or force to the stylet 400 by, for example, pushing down on the stylet 400 to use the inclined surface 440 on the needle head 406 to move the push button 316 into an unlocked position. This allows the surgeon to press down on the stylet 400 to move the push button 316 into the first groove 414 on the lower part 412 of the needle head 406 without having to press on the push button 316 itself. In this way, the surgeon may be able to initially lock the stylet 400 at a first depth without having to press on the push button 316.

In some embodiments, the surgeon may press the push button 316 to move it into an unlocked position. While pressing the push button 316, the surgeon may then move the stylet 400 to the desired depth. The surgeon may reference the measurement indicators 426, 428, 430, 432, 434 to determine the desired depth, where the depth corresponds to the length of the exposed length 411 the tip 410 of the needle 408 extending from the bottom 218 of the screw 216. The surgeon may then align the line 436 of desired measurement indicator 426, 428, 430, 432, 434 with the top 302 of the carrier 300 and release the push button 316. The push button 316 can then contact the groove 414, 416, 418, 420, 422 that corresponds to the desired measurement indicator 426, 428, 430, 432, 434. Thus, the exposed length 411 of the needle 408 will match the desired depth.

Once the push button 316 contacts the desired groove 414, 416, 418, 420, 422, the stylet 400 cannot move up or down in reference to the carrier 300 unless the surgeon pushes the push button 316 to move it into the unlocked position.

In one particular example, the surgeon may want to drive the needle 408 at a depth of 10 mm, which may mean that the exposed length 411 of the needle 408 should be 10 mm. Thus, the surgeon pushes the push button 316 and moves the stylet 400 (or, in some embodiments, the carrier 300, handle 500, screwdriver 600, and pedicle screw assembly 200) so that the line 436 of the measurement indicator 428 corresponding to 10 mm aligns with the top 302 of the carrier 300. The surgeon may release the push button 316 so that it contacts the groove 416 corresponding to 10 mm, locking the stylet 400 relative to the carrier 300, thereby resulting in an exposed length 411 of 10 mm.

Referring back to FIG. 6, step 730 includes positioning the inserter device 100. The surgeon may place the tip 410 of the needle 408 at the position where the surgeon would like to implant the pedicle screw assembly 200. The surgeon may choose the desired location of the pedicle screw system 200 and the desired angle at which the pedicle screw system 200 is implanted.

At step 740 in FIG. 6, the tip 410 of the stylet 400 may be driven into bone. Once the surgeon places the inserter device 100 at the desired position, the tip 410 of the needle 408 may be inserted in that desire position. The surgeon may drive the tip 410 into bone by any appropriate method. For example, the surgeon may tap the top 402 of the stylet 400. In some embodiments, the surgeon may rotate the inserter device 100 to drive the tip 410 into bone.

The surgeon may drive the tip 410 into bone until the entire exposed length 411 is driven into the bone. In other words, the surgeon may drive the tip 410 into bone until the surface of the bone contacts the bottom 218 of the screw 202. In some embodiments, the bottom 218 of the screw 202 will physically prevent the surgeon from inserting the inserter device 100 further into the bone.

At step 750 of FIG. 6, the surgeon may optionally adjust the inserter device 100 to a second exposed length 411 or depth. If the surgeon decides to implant the needle 408 deeper into the bone than the first depth (which corresponds to the exposed length 411), then surgeon can adjust the inserter device 100 to a second elongate length 411 corresponding to the second depth. In some embodiments, the surgeon may leave the part of the needle 408 driven into bone in the bone while adjusting the inserter device 100 to the second depth. In this case, the surgeon can press the push button 316 into an unlocked position. The surgeon can hold the stylet 400 steady while moving the carrier 300, handle 500, screwdriver 600, and pedicle screw assembly 200 up the stylet 400 until the top 302 of the carrier 300 aligns with the line 436 of the desired measurement indicator 426, 428, 430, 432, 434. The surgeon can then release the push button 316 so that it contacts the groove 414, 416, 418, 420, 422 that corresponds to the desired measurement indicator 426, 428, 430, 432, 434, thereby moving the push button 316 into a locked position and locking the stylet 400 relative to the carrier 300.

In some embodiments, the surgeon can remove the needle 408 from the bone, readjust the second depth, then drive the exposed length 411 of the needle 408 back into the bone through the same hole or through a new hole.

In some embodiments, the surgeon may want to implant the needle 408 at a shorter depth than the first depth. In this case, the surgeon may need to pull the inserter device 100 back to pull some or all of the exposed length 411 of the needle 408 out of the bone before adjusting the inserter device 100. Moreover, the surgeon may need to move the carrier 300, handle 500, screwdriver 600, and pedicle screw assembly 200 down the stylet 400 rather than up.

In a particular example, the surgeon may have initially driven an exposed length 411 of 10 mm into the bone, but may decide to drive the needle 408 an additional 5 mm into the bone. The surgeon can then press the push button 316 to move it into an unlocked position and move the assembled carrier 300, handle 500, screwdriver 600, and pedicle screw assembly 200 up the stylet 400 until the top 302 of the carrier 300 aligns with the line 436 of the measurement indicator 430 that corresponds to 15 mm. Then, the surgeon can release the push button 316 so that it contacts the groove 418 corresponding to an exposed length 411 of 15 mm. Thus, the surgeon has 5 mm of additional exposed length 411 to drive into the bone.

Referring back to FIG. 6, at step 760, the surgeon may drive the needle 408 further into the bone. In some embodiments described above, the surgeon has lengthened the exposed length 411 while the tip 410 is still implanted in the bone. Thus, the surgeon may continue to drive the additional exposed length 411 into bone through any appropriate method, including, for example, tapping the top 402 of the stylet 400 or rotating the stylet 400. In some embodiments, the surgeon may remove the first exposed length 411 that had been driven into the bone before adjusting the inserter device 100 to the second exposed length 411. In these embodiments, the surgeon may reposition the inserter device 100 to the same position or to a different position. The surgeon may then proceed to drive the second exposed length 411 into the bone by any appropriate method, including, for example, tapping the top 402 of the stylet 400 or rotating the stylet 400.

As with step 740 described above, in step 760 the surgeon may drive the tip 410 into bone until the entire second exposed length 411 is driven into the bone. In other words, the surgeon may drive the tip 410 into bone until the surface of the bone contacts the bottom 218 of the screw 202. In some embodiments, the bottom 218 of the screw 202 will physically prevent the surgeon from inserting the inserter device 100 further into the bone.

In some embodiments, steps 750 and 760 may be repeated as many times as desired to drive the needle 408 to the desired depth in the bone. In some embodiments, only steps 730 and 740 are performed and steps 750 and 760 are not performed.

Referring back to FIG. 6, at step 770, the screw 202 of the pedicle screw assembly 200 may be inserted into the bone around the exposed length 411 of the needle 408 that has been driven into the bone. In some embodiments, the surgeon may rotate the handle 500 thereby screwing the threading 220 of the shaft 216 of the screw 202 into the bone.

As described above, the thread pitch of the threading 220 of the screw 202 may be equal to the thread pitch of the threading 314 on the lower part 310 of the carrier 300. Thus, when the surgeon rotates the handle 500 to screw the screw 202 into the bone, screw 202 advances (along with the handle 500, screwdriver 600, and pedicle screw assembly 200) into the bone at the same rate as the carrier 300 (along with the stylet 400) screws out and up from the handle 500. This occurs because the thread pitch of the threading 220 on the screw 202 is the same as the threading 314 on the lower part 310 of the carrier 300. This prevents the needle 408 from driving further into the bone as the screw 202 is being screwed into the bone around the needle 408. Thus, the needle 408 will not be driven into the bone to an undesired depth and contact, for example, a nerve.

Similarly, in other embodiments, the thread pitch of the threading 220 of the screw 202 may be greater than the thread pitch of the threading 314 on the lower part 310 of the carrier 300. Thus, when the surgeon rotates the handle 500 to screw the screw 202 into the bone, screw 202 advances (along with the handle 500, screwdriver 600, and pedicle screw assembly 200) into the bone while the carrier 300 (along the stylet 400) screws out and up from the handle 500 at a greater rate. In this way, the exposed length 411 of the needle 408 will not be driven further into the bone and will instead back out of the bone. This occurs because the thread pitch of the threading 220 on the screw 202 is greater than that of the threading 314 on the lower part 310 of the carrier 300. This prevents the needle 408 from driving further into the bone as the screw 202 is being screwed into the bone around the needle 408. Thus, the needle 408 will not be driven into the bone to an undesired depth and contact, for example, a nerve.

Referring back to FIG. 6, at step 780, the inserter device 100 may be removed from the pedicle screw assembly 200. In some embodiments, the grip 636 of the tulip connector 602 of the screwdriver 600 may be rotated to unscrew the threaded portion 618 of the tulip connector 602 from the threading 211 on the towers 210 of the tulip 204. When the tulip connector 602 and the pedicle screw assembly 200 are no longer threadably engaged, the surgeon may slide the screwdriver 600 up and out of the towers 210 by sliding the extensions 632 of the winged portion 630 along the channel 224 between the tower 210.

In some embodiments, the stylet 400 may be removed before the screwdriver 600 is removed from the pedicle screw assembly 200. In some embodiments, the assembled singe step inserter 100 may be removed at once. In some embodiments, the stylet 400, the carrier 300, and/or the handle 500 may be removed from the screwdriver 600 before the screwdriver 600 is removed from the pedicle screw assembly 200.

The towers 210 may also be removed from the tulip 204 along the intersection 212 between the tower 210 and the arms 206 of the tulip 204. In some embodiments, the towers 210 may be removed while they are still threadably engaged with the tulip connector 602. In other embodiments, the tulip connector 602 and the rest of the inserter device 100 may be removed before the towers 210 are removed. The towers 210 may be removed in any appropriate way. For example, the towers 210 can be broken from the arms 206 at the intersection 212, a tool can be used to cut or remove the intersection 212, or the towers 210 can be removed from the arms 206 in any other appropriate way. In other embodiments, the towers 210 may remain on the tulip 204 until a rod is disposed and secured between the arms 206 of the tulip 204.

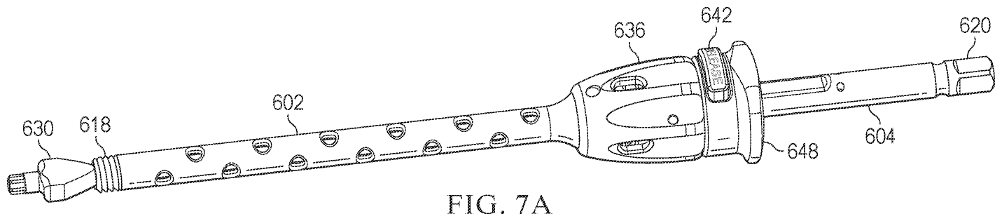

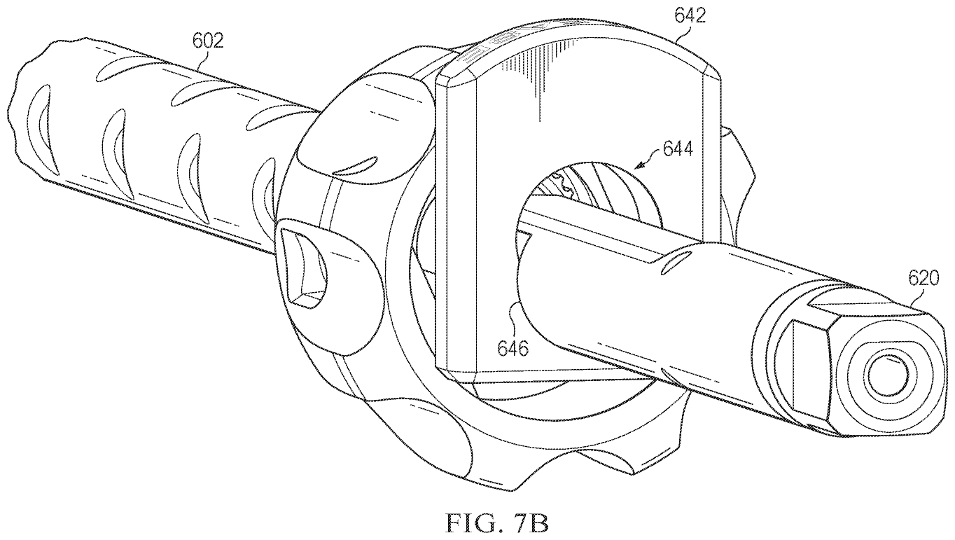

FIGS. 7A-7B show another embodiment of the screwdriver 600, according to some aspects of the present disclosure. FIG. 7A is a side view of the screwdriver 600 and FIG. 7B is a top view of the screwdriver 600 with the lock housing 648 removed (as described in more detail below). The screwdriver 600 shown in FIGS. 7A-7B may be similar to the screwdriver shown in FIGS. 1A-1B and 5A-5C. In the embodiment of FIGS. 7A-7B, the tulip connector 602 comprises a lock that locks the screw connector 604 to the tulip connector 602. The lock may be disposed at the top 606 of the tulip connector 602 and may be coupled thereto such that the screw connector 604 can pass through the opening 610 of the tulip connector 602 and the lock. The lock comprises a lock housing 648 and a push button 642 disposed within the lock housing 648. The push button 642 extends through the lock housing 648 from one side and passes through the opening 610. The push button 648 has an aperture 644. The push button 648 is able to slide within the lock housing 648 such that the aperture 644 can align with the opening 610 in a first (i.e. unlocked) position and can be offset from the opening 610 in a second (i.e. locked) position. In some embodiments, there may be a biasing mechanism disposed in the lock housing 648 that biases the push button 642 towards the locked position. The surgeon can press the push button 642 to move the push button 642 so that the aperture 644 is aligned with the opening 610. In this way, the surgeon can move the push button 642 into the unlocked position by pressing on the push button 642. Any appropriate biasing mechanism may be used, including the biasing mechanism described above in relation to the carrier 300.

The push button 642 may comprise a locking surface configured to engage a corresponding groove, slot, or recessed portion 646 on the screw connector 604. In the default, or resting position, the locking surface of the push button 642 may be biased to engage the recessed portion 646 on the screw connector 604 such that the screw connector 604 is locked in at least the longitudinal direction with respect to the tulip connector 602. Depressing the push button 642 inward may cause the locking surface of the push button 642 to disengage the recessed portion 646 so that the screw connector 604 can be retracted from the opening 610. Although the illustrated embodiment has a lock including a push button 642, any appropriate locking mechanism may be used.

In some embodiments, the inserter device 100 is provided as part of a kit. The kit may include the inserter device 100 and one or more pedicle screw assemblies 200. The inserter device 100 may be provided assembled or may be provided in parts for the surgeon to assemble during a procedure. Similarly, the pedicle screw assemblies 200 may be provided assembled or may be provided in parts for the surgeon to assemble during a procedure. In some embodiments, the kit may have one type of tulip 204 and various types and lengths of screws 202. In some embodiments, the kit may comprise two or more stylets 400 of various lengths and with various measurement indicators. A particular length of the stylet 400 may be paired with a particular length of screw 202, so the kit may include various screws 202 and their respective stylets 400.

While various embodiments in accordance with the disclosed principles have been described above, it should be understood that they have been presented by way of example only, and are not limiting. Thus, the breadth and scope of the invention(s) should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

What is claimed is:

1. A device for implanting a screw into a bone of a patient comprising:
a carrier comprising:
a first portion;
a threaded second portion opposite the first portion;
an opening extending from the first portion to the second portion, wherein the opening is configured to receive a stylet; and
a lock, wherein the lock is in communication with the opening and can be moved between a locked position and an unlocked position, wherein the lock engages the stylet disposed within the opening to lock a position of the stylet relative to the carrier;
a handle having a threaded portion, wherein the second portion of the carrier threadably engages with the threaded portion of the handle; and,
a screwdriver coupled to the handle and configured to engage a threaded screw, wherein the screwdriver comprises a lumen configured to receive the stylet.

2. The device of claim 1, wherein a first thread pitch of the threaded second portion of the carrier is greater than a second threaded pitch of the threaded screw.

3. The device of claim 1, wherein a first thread pitch of the threaded second portion of the carrier is equal to a second threaded pitch of the threaded screw.

4. The device of claim 1, wherein the stylet comprises an elongate needle and a needle head, wherein the needle head comprises plurality of grooves.

5. The device of claim 4, wherein the lock engages at least one of the plurality of grooves when in the locked position.

6. The device of claim 4, wherein the plurality of grooves are arranged vertically along a length of the needle head.

7. The device of claim 6, wherein each groove of the plurality of grooves corresponds to a length a tip of the needle extends past a bottom of the screw.

8. A device for implanting a screw into bone comprising:
a carrier comprising:
a first portion;
a threaded second portion;
an opening extending from the first portion to the second portion; and
a lock moveable between a locked position and an unlocked position;
a stylet having an elongate needle and a needle head, wherein at least a portion of the needle head is disposed within the carrier, wherein the lock engages the needle head in the locked position;
a handle having a third portion and a fourth portion, wherein the third portion of the handle is threadably engaged with the threaded second portion of the carrier;
a screwdriver coupled to the fourth portion of the handle; and,
a screw coupled to the screwdriver.

9. The device of claim 8, wherein a first thread pitch of the threaded second portion of the carrier is greater than a second threaded pitch of the threaded screw.

10. The device of claim 8, wherein a first thread pitch of the threaded second portion of the carrier is equal to a second threaded pitch of the threaded screw.

11. The device of claim 8, wherein the stylet is moveable within the carrier when the lock is in the unlocked position.

12. The device of claim 8, wherein the needle head comprises a plurality of grooves.

13. The device of claim 12, wherein the lock engages at least one of the plurality of grooves when in the locked position.

14. The device of claim 12, wherein the plurality of grooves are arranged vertically along a length of the needle head.

15. The device of claim 12, wherein each groove of the plurality of grooves corresponds to a length a tip of the needle extends past a bottom of the screw.

16. A method of implanting a screw into a bone of a patient, comprising the steps of:
providing an instrument comprising:
a carrier defining an opening configured to receive a stylet, the carrier comprising a lock moveable between a locked position and an unlocked position;
a handle having a third portion and a fourth portion, wherein the third portion of the handle is threadably engaged with the carrier;
a screwdriver coupled to the fourth portion of the handle;
inserting the stylet into the instrument, the stylet having an elongated needle and a needle head, wherein the elongate needle extends through the instrument and the screw, and wherein at least a portion of the needle head is disposed within the carrier and the lock engages the needle head such that a first length of a tip of the needle extends past a distal end of the screw;
implanting the first length of the tip of the needle into the bone; and
implanting the screw into bone.

17. The method of claim 16, further comprising the steps of:
adjusting, after implanting the first length of the tip of the needle into the bone, the instrument so that the needle extends a second length past the distal end of the screw; and,
implanting the second length of the tip of the needle into the bone.

18. The method of claim 17, wherein adjusting the instrument comprises moving the lock of the carrier into an unlocked position.

19. The method of claim 17, wherein the needle head of the stylet comprises a plurality of grooves.

20. The method of claim 19, wherein adjusting the instrument comprises:
moving the lock into an unlocked position;
moving the carrier relative to the stylet such that the lock aligns with a groove of the plurality of grooves; and,
moving the lock into a locked position, wherein the lock engages the groove of the plurality of grooves.
